(19) United States
(12) Patent Application Publication
(10) Pub. No.: US 2012/0224664 A1
(43) Pub. Date: Sep. 6, 2012

(54) TOMOSYNTHESIS MAMMOGRAPHY SYSTEM WITH ENLARGED FIELD OF VIEW

(75) Inventor: Hanns-Ingo Maack, Norderstedt (DE)

(73) Assignee: KONINKLIJKE PHILIPS ELECTRONICS N.V., EINDHOVEN (NL)

(21) Appl. No.: 13/509,422

(22) PCT Filed: Nov. 17, 2010
(86) PCT No.: PCT/IB2010/055213

§ 371 Date: May 11, 2012

(30) Foreign Application Priority Data
Nov. 20, 2009 (EP) 09176611.3

(51) Int. Cl. A61B 6/04; A61B 6/03

(52) U.S. Cl. 378/7; 378/19

(57) ABSTRACT

A tomosynthesis system for acquiring a three-dimensional image of an object such as a mammography image of a female breast is proposed. The tomosynthesis system (1) comprises an X-ray source (3), an X-ray detector (7), a support arrangement (15) and a moving mechanism (11). The X-ray source (3) and the X-ray detector (7) are adapted for acquiring a plurality of X-ray images while irradiating the object (17) with an X-ray beam (21) from a plurality of tomographic angles α. The moving mechanism (11) is adapted to pivot the X-ray detector (7) in positions such that for each tomographic angle α a detection surface (25) of the X-ray detector (7) is oriented to be substantially perpendicular to the X-ray beam (21). The moving mechanism (11) is adapted to move the X-ray detector (7) in positions such that a distance between the X-ray source (3) and the detector (7) is increased with increasing tomographic angle a thereby enabling that the X-ray detector (7) remains within an enlarged housing (5) during an entire tomographic image acquisition procedure.

### FIELD OF THE INVENTION

The present invention relates to a tomosynthesis system for generating a three-dimensional image of an object such as a three-dimensional mammography image of a female breast.

### BACKGROUND OF THE INVENTION

In order to detect and analyze breast cancer, various mammography systems are known.

In conventional mammography screening systems, the female breast is compressed between two plates and soft X-rays are transmitted through the compressed tissue before being detected by an X-ray detector. However, planar mammography is inherently limited to representing 3D information in a 2D plane. While high lateral resolution, i.e. in an x-y-plane, may be achieved, no depth resolution, i.e. in a z-direction, may be obtained.

In order to also realize depth resolution and furthermore in order to relax the requirement of strongly compressing the breast during examination, tomosynthesis mammography systems, also referred to as digital breast tomosynthesis (DBT) systems, have been developed. In these systems, a plurality of X-ray images may be acquired while the breast is irradiated with an X-ray beam from a plurality of tomographic angles. Conventionally, an X-ray source is moved along a circular arc path while being always oriented towards a fixed detector above which the breast is supported. Conventionally, X-ray images are acquired within a maximum range of tomographic angles of up to 2×25°. From the plurality of acquired two-dimensional X-ray images, a final three-dimensional image of the breast may be generated. Such three-dimensional image may provide for both, good lateral resolution and sufficient depth resolution, wherein the depth resolution typically increases reciprocally proportional with the range of tomographic angles (1/α).

Another alternative for mammography examination is breast computer tomography (CT). Therein, a patient is lying with her breast through a hole in a prone table. While the breast is substantially not compressed, an X-ray imaging system comprising an X-ray source and an opposite X-ray detector is rotated horizontally around the breast and more than 100 projection X-ray images are taken within a large tomographic angle range (>180°). However, the X-ray tube voltage is typically much higher than for conventional mammography systems (typically >49 kV). Therefore, an X-ray sensitive layer of the detector has to be typically thicker, leading to a worse lateral resolution. The depth resolution may be much higher than in digital breast tomosynthesis systems. Accordingly, the spatial resolution may quite anisotropic. Typically a contrast agent is injected for the examination, so this modality may be not well suited for screening examinations.

### SUMMARY OF THE INVENTION

There may be a need for an improved tomosynthesis mammography system which enables high spatial image resolution and/or a large field of view while preferably providing improved patient comfort.

According to an aspect of the present invention, a tomosynthesis system for generating a three-dimensional image of an object such as a mammography image of a female breast is suggested. The system comprises an X-ray source, an X-ray detector, a support arrangement and a moving mechanism. The X-ray source and the X-ray detector are adapted for acquiring a plurality of X-ray images while irradiating the object with an X-ray beam from a plurality of tomographic angles α. The support arrangement is adapted to support the object during operation of the tomosynthesis system. The moving mechanism is adapted to pivot the X-ray detector in positions such that for each tomographic angle α the detection surface of the X-ray detector is oriented to be substantially perpendicular to the incident X-ray beam. Furthermore, the moving mechanism is adapted to move the X-ray detector in positions such that a distance (SID (source image distance)) between the X-ray source and the detector increases with increasing tomographic angle α.

A gist of the suggested tomosynthesis system may be seen as based on the following findings and ideas: In conventional digital breast tomosynthesis systems, while an X-ray source is moved along an arcuate path in order to irradiate an object to be observed from a plurality of tomographic angles, the X-ray detector is conventionally fixed in space. While this may allow for a simple moving mechanism which only has to move the X-ray source, a resulting three-dimensional field of view may be reduced when compared to normal screening mode mammography imaging. Furthermore, due to the X-ray detector being fixed, an X-ray beam from the X-ray source impinges onto the X-ray detector perpendicular only for a 0°-position of the X-ray source. At any other tomographic angles α≠0°, the X-ray beam will impinge onto the X-ray detector's surface under the corresponding angle α possibly resulting in the fact that not all X-rays may impinge onto the detection surface and may be detected by the detector. This may limit a possible range of tomographic angles to less than 25° (α≦25°).

In order to overcome such limitations, it is proposed herein to provide the tomosynthesis system with a moving mechanism such that not only the X-ray source may be displaced in order to irradiate under various tomographic angles α but also the X-ray detector may be displaced in a specific way. Specifically, the moving mechanism is adapted to pivot the X-ray detector in such a way that an X-ray beam from the X-ray source always impinges onto the detection surface of the X-ray detector perpendicularly. In other words, while the X-ray source may be positioned at various locations along an arcuate path in order to irradiate the object to be examined from various tomographic angles α, a positioning of the X-ray detector is adjusted such that, independent of the selected tomographic angle α, the X-ray beam is perpendicular to the detection surface of the detector. Therein, “perpendicular” may mean that a direction of the X-ray beam is normal to a plane of the detection surface and that a middle axis of the X-ray beam crosses the detection surface on a center axis thereof. For mammography applications, the middle axis of the X-ray beam usually crosses the detection surface not in a center point thereof but somewhere on the center axis close to an edge of the detection surface in order to be able to also acquire images of breast tissue close to the thorax of the patient. While the X-ray source may be moved along a circular arc and is always oriented with a center axis of the X-ray beam being directed towards the center of the circular arc, the X-ray detector may be displaced with a rather complex motion. For example, for a 0°-position of the X-ray source, the X-ray detector may be positioned centrally underneath the support arrangement for supporting the object such that the center of the detection surface substantially coincides with the center of the circular arcuate path. In such 0°-position, the distance between the X-ray source and the detector is minimum. In this 0°-position, the source-detector arrangement essentially corresponds to an arrangement as used for conventional mammography screening applications.

For position of the X-ray source outside the center of the circular arcuate path, i.e. α>0°, the X-ray detector is moved off-center. It is to be noted that the X-ray detector is not only rotated about for example its symmetry axis but is pivoted, i.e. a rotary movement is combined with a translational movement. Such pivoting motion may be selected such that, while the X-ray detector is always rotated so as to be oriented towards the X-ray source, the X-ray detector is at the same time moved translational in order to provide for the X-ray detector always remaining underneath the support arrangement supporting the object to be examined. Such translational movement may be chosen such that the distance (SID) between the X-ray source and the X-ray detector increases with increasing tomographic angle.

For example, the SID may be proportional to the tangent of the tomographic angle α, i.e. SID=a * tan (α), with a being a constant.

According to an embodiment of the present invention, the proposed tomosynthesis system further comprises a housing enclosing the X-ray detector. Therein, dimensions of the housing are sized such that and the moving mechanism is adapted such that for all positions to which the X-ray detector may be moved by the moving mechanism, the housing encloses the X-ray detector. In other words, in contrast to conventional systems where the X-ray detector is accommodated in a housing being only slightly larger than the detector itself, it is proposed herein to provide a housing for the X-ray detector, the housing being substantially larger than the X-ray detector. Thus, the X-ray detector may be moved and pivoted within the housing in a way such as to fulfil the above described conditions of e.g. perpendicular X-ray beam incidence. Specifically, the housing and the motion of the X-ray detector being guided by the moving mechanism are adapted such that for all possible angular positions of the X-ray source, the detector is oriented perpendicular to the incoming X-rays and remains entirely within the housing.

According to an embodiment, the housing comprises a flat or concave surface forming the support arrangement for supporting the object to be examined. In other words, the housing of the X-ray detector may not only serve as a protection for the detector but may also serve for supporting the object, i.e. e.g. the female breast.

Preferably, the flat or concave surface of the housing forms the only X-ray absorption surface within an optical path between the X-ray source and the X-ray detector. In other words, in the proposed tomosynthesis system, the X-ray detector is comprised in such large housing that the flat or concave surface of the housing supporting the examined object is the only material layer within the X-ray beam (apart from the object itself) absorbing X-rays.

An alternative would be to have no such housing both enclosing the detector and supporting the object but instead move the detector free in the air and supporting/compressing the object between separate support/compression plates. In such case, the detector would need its own covering housing and furthermore, the support arrangement would need a supporting surface such that at least two X-ray absorbing material layers would have to be provided within the X-ray beam. Due to the fact that any material layer (made for example from carbon fibre) has about 15% X-ray absorption, an additional material layer would lead to a DQE drop (detective quantum efficiency) of the system in the same order of magnitude.

According to a further embodiment, the moving mechanism is of the proposed tomosynthesis system is adapted to pivot and move the X-ray detector such that for all tomographic angles α one edge of the X-ray detector is positioned adjacent to the flat or concave surface of the housing. In other words, the moving mechanism may move the X-ray detector such that, while fulfilling the above-mentioned conditions of inter alia perpendicular incidence, the X-ray detector is always maximally close to the surface of the housing supporting the examined object.

According to a further embodiment, the housing comprises a flexible front cover. Therein, the front cover may be a surface of the detector housing being directed towards a patient standing with her breast lying on the supporting surface of the housing. Due to the front cover being flexible, it may be deformed during for example a screening examination when being in mechanical contact e.g. with a belly of a heavy woman.

According to a further preferred embodiment, the proposed tomosynthesis system comprises an anti-scatter-grid arrangeable between the X-ray detector and the support arrangement. Such anti-scatter-grid may be provided for attenuating scattered X-rays thereby enabling an improved signal-to-noise ratio of the acquired X-ray images. The anti-scatter-grid may comprise X-ray absorbing walls being oriented parallel to X-rays of an X-ray beam impinging perpendicular onto the detection surface of the X-ray detector. In conventional tomosynthesis systems having a fixed detector, no such anti-scatter-grid may be used as the X-ray beams impinge under various angles onto the X-ray detector depending on the selected tomographic angle α such that an anti-scatter-grid being specifically adapted for one specific angle of incidence would be non-optimum for all other angles of incidence. In contrast hereto, as according to the present invention, the X-ray detector is always positioned such as to orient perpendicular to incoming X-rays, an anti-scatter-grid being adapted for such perpendicular incidence may be suitable for all tomographic angles α.

Specifically, the anti-scatter-grid may be mechanically connected to the X-ray detector. Accordingly, the anti-scatter-grid may be moved together with the X-ray detector by the moving mechanism so as to be oriented in an optimum way towards the X-ray source. However, for some applications, the provision of an anti-scatter grid within the beam path may not be desired. Accordingly, there may be a grid displacement mechanism which may displace the grid into a parking position outside the beam path.

Furthermore, a grid moving mechanism may be provided for moving the anti-scatter-grid parallel to the detection surface of the X-ray detector. Such movement of the anti-scatter-grid may avoid the formation of stripes within the acquired X-ray image. Typically, a linear movement may be in a range in the order of 2 cm. When the anti-scatter-grid is in an extreme position, it may be stopped and moved in the reverse direction.

According to another embodiment of the proposed tomosynthesis system, the moving mechanism is further adapted to move the detector such as to increase the distance (SID) between the X-ray source and the detector while an orientation of the detector remains fixed. In other words, additional to a first motion mode as described above in which the X-ray detector is moved in a pivoting motion in order to be always oriented towards the X-ray source, the moving mechanism also enables a second motion mode in which only the distance between the X-ray source and the detector is varied while the X-ray detector is not rotated/pivoted. Such possibility of varying the source-detector distance SID may enable a suitable magnification of the acquired X-ray image so that spatial resolution and DQE may be improved for example when acquiring images of a small breast. In such application, the provision of an anti-scatter grid may not be desired as the anti-scatter grid is usually optimized for one specific source-detector distance SID. Accordingly, the anti-scatter grid may be displaced into the parking position outside the beam path.

With the proposed tomosynthesis system, the X-ray source and the X-ray detector may be adapted to acquire X-ray images within a range of tomographic angles of more than +/−25°, for example more than +/−45°, preferably up to +/−60°. Such increased acquisition range may be mainly due to the fact that the X-ray detector is always oriented towards the X-ray source. Accordingly, even at high tomographic angles, no significant image distortion may occur. Furthermore, even at such high tomographic angles, an anti-scatter-grid may be used in order to improve a signal-to-noise ratio

With the proposed tomosynthesis mammography system, tomographic angles larger than 45° may be feasible, leading to better depth resolution combined with high 2D-sharpness. The proposed tomosynthesis system is compatible with conventional geometries and allows for both, regular screening mode and tomosynthesis mode. Furthermore, also stereotactic (guided) biopsy may be possible. Particularly for heavy breasts, a better contrast resolution may be obtained due to the possible use of an anti-scatter-grid. Furthermore, for small breasts, a variable source-detector distance may allow to use magnification techniques which also may lead to improved image quality.

It is to be noted that aspects and embodiments of the present invention are described herein partly with respect to the tomosynthesis system and its structural or functional features and partly with respect to a possible mode of use of such tomosynthesis system. However, a person skilled in the art will gather from the above and the following description that, unless other notified, in addition to any combination of features belonging to one type of description also any combination between features relating to different embodiments is considered to be disclosed with this application.

### BRIEF DESCRIPTION OF THE DRAWINGS

Features and advantages of the present invention will be further described with respect to specific embodiments as shown in the accompanying drawings to which the invention shall not be limited.

FIG. 1 shows a side view of a tomosynthesis system according to an embodiment of the present invention.

FIG. 2 schematically indicates varying positions of an X-ray detector in a front view of a tomosynthesis system according to an embodiment of the present invention at different tomographic angles.

FIG. 3(a)-(c) schematically illustrate a pivoting movement of an X-ray detector for a tomosynthesis system according to an embodiment of the present invention.

FIG. 4 shows a graph illustrating an increase of a source-detector distance SID depending on a tomographic angle.

FIG. 5 illustrates a housing for an X-ray detector for a tomosynthesis system according to an embodiment of the present invention.

FIG. 6 illustrates a housing for an X-ray detector for a tomosynthesis system according to another embodiment of the present invention.

FIG. 7 illustrates an arrangement to acquire off-center screening images in a tomosynthesis system according to an embodiment of the present invention.

FIG. 8 illustrates a magnification mode with a displaced X-ray detector in a tomosynthesis system according to an embodiment of the present invention.

FIG. 9 illustrates an X-ray detector with an anti-scatter-grid for use in a tomosynthesis system according to an embodiment of the present invention.

FIG. 10 illustrates the tomosynthesis system of FIG. 1 wherein the housing of the X-ray detector has a flexible front cover.

FIG. 11 shows a flow-chart of an operating method of a tomosynthesis system according to an embodiment of the present invention.

All figures are only schematically and not to scale. Similar features are indicated with similar or same reference signs throughout the figures.

### DETAILED DESCRIPTION OF EMBODIMENTS

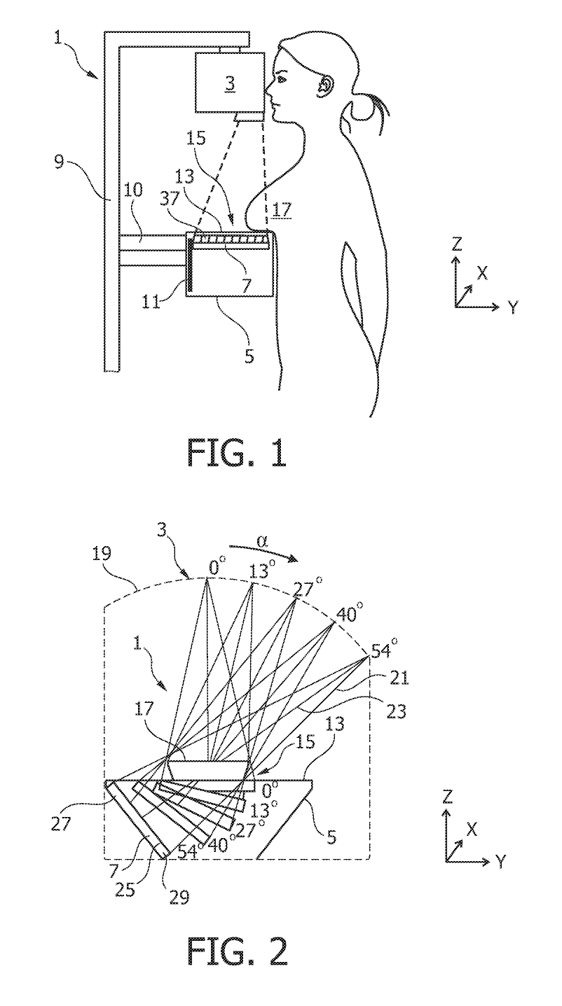

FIG. 1 shows a side view of a tomosynthesis mammography system 1 according to an embodiment of the present invention. An X-ray source 3 and a housing 5 comprising an X-ray detector 7 are attached to a supporting frame 9. An upper surface 13 of the housing 5 acts as a support arrangement 15 for supporting the female breast 17 to be examined during the operation of the tomosynthesis system 1. The housing 5 is substantially larger, for example by a factor 1.5 to 5, in its x-direction and its z-direction than the X-ray detector 7 accommodated therein. For example, the housing may be up to three times as large as the X-ray detector 7 in the x-direction and up to 5 times as large in the z-direction. Accordingly, the X-ray detector 7 may be arranged within the housing 5 at different locations and in different orientations. The housing 5 also comprises a moving mechanism 11 which is adapted to move the detector 7 along a pivoting motion path. Furthermore, as will be described further below, the detector 7 may be provided with an anti-scatter grid which, when its use is not desired, may be displaced into a parking position within an extension 10 of the housing 5.

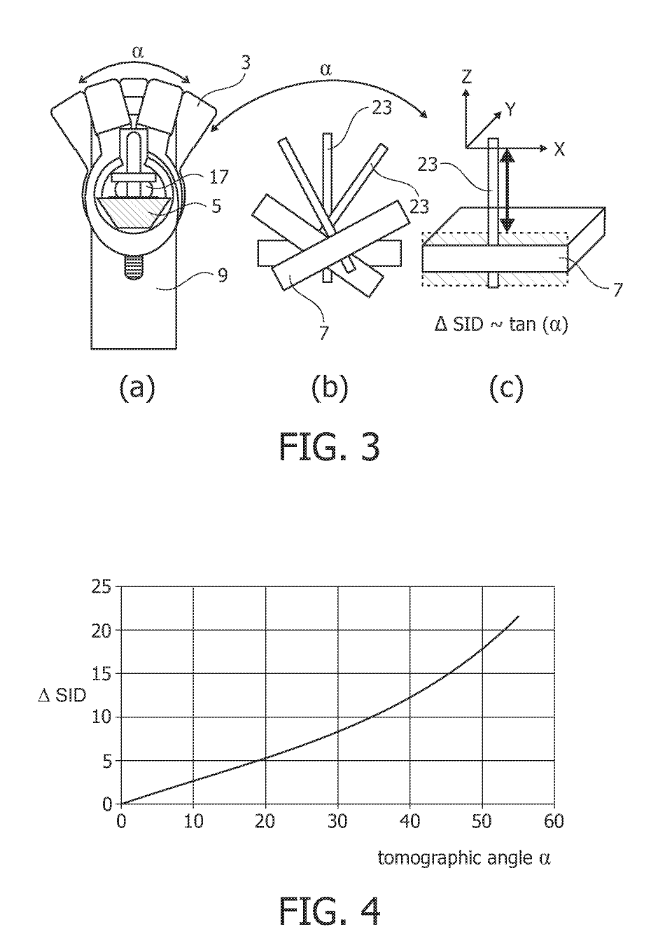

In the front views shown in FIG. 2 and FIG. 3, the pivoting motion of the X-ray detector 7 within the housing 5 is schematically illustrated. The X-ray source 3 may be arranged at various locations along an arcuate path 19 in order to irradiate the female breast 17 under a plurality of tomographic angles α. Together with the motion of the X-ray source 3 also the X-ray detector 7 is moved within the housing 5 guided by the moving mechanism 11. Therein, depending on the prevailing tomographic angle α, which is shown to be in a range of 0° to 54°, the detector 7 is pivoted into such an orientation that an X-ray beam 21 coming from the X-ray source 3 impinges with its center axis 23 perpendicular to a detection surface 25 of the X-ray detector 7.

As indicated in FIG. 3(b) and FIG. 3(c), the pivoting motion of the detector 7 can be interpreted as a superposition of

(i) a rotational motion around the y-direction in which rotational motion the detector 7 is rotated to an orientation corresponding to the prevailing tomographic angle α (FIG. 3(b)), and
(ii) a radial motion in which a distance SID between the X-ray source 3 and the X-ray detector 7 along the middle axis 23 of the X-ray beam 21 is changed depending on the prevailing tomographic angle α. Accordingly, the moving mechanism may be adapted for guiding two motion components, one motion component being a rotation around the y-direction and one motion component being a radial translation normal to the detector's surface. Therein, the change of the source-detector distance ΔSID may be proportional to the tangent of the tomographic angle α as indicated in FIG. 4. However, particularly for small tomographic angles, the dependency between the change of the source-detector distance ΔSID and the tomographic angle α may also follow another function; for example, there may be a linear or polynomial increase of ΔSID with the tomographic angle α.

In order to pivot the detector 7 as shown in FIG. 2, the moving mechanism 11 may be adapted to both, rotate the detector 7 around the y-axis and to translate the X-ray detector 7 along a direction normal to its detection surface 25. Therein, the X-ray detector 7 shall be rotated and translated such that it is always oriented towards the X-ray source 3, i.e. arranged with its normal axis corresponding to the tomographic angle α, and such that the X-ray detector 7 remains within the housing 5, i.e. does not hit any walls of the housing 5.

Advantageously, as shown in FIG. 2, the X-ray detector 7 is pivoted such that in each angular position, it remains as close as possible to the supporting upper surface 13 while fulfilling the previously mentioned conditions. This means that one edge 27 of the X-ray detector 7 remains adjacent to the supporting surface 13 while an opposing edge 29 of the X-ray detector 7 moves along an arcuate path into the depth of the housing 5 while the X-ray detector 7 is arranged in order to correspond to the tomographic angle α.

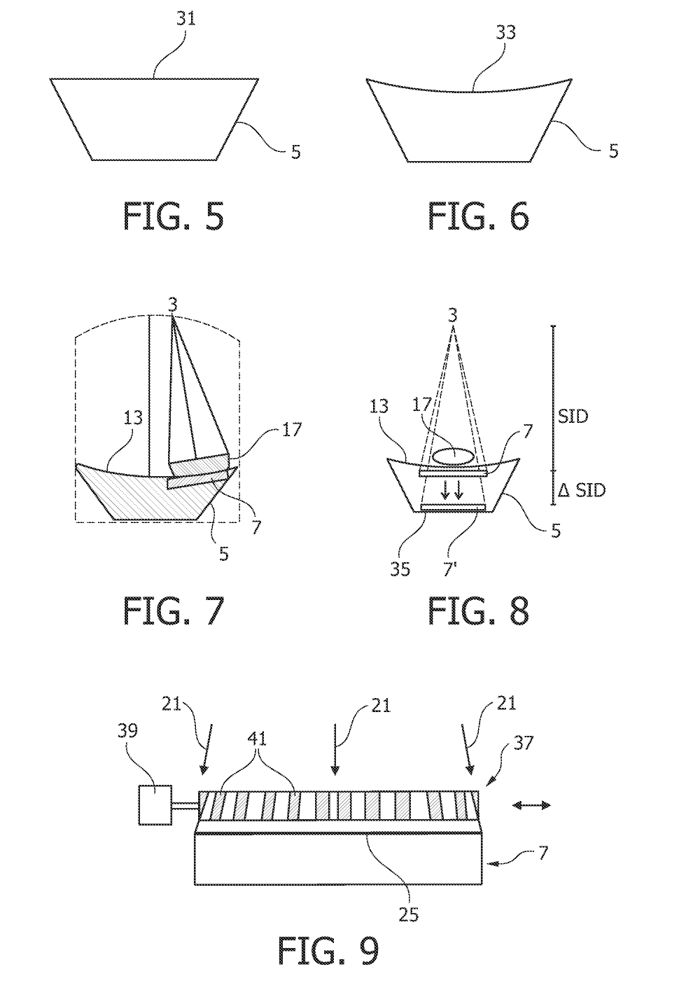

As shown in FIG. 5, the housing 5 may have a flat upper surface 31 acting as support arrangement for the female breast 17 to be deposited thereon during mammography imaging. Alternatively, as shown in FIG. 6, the housing 5 may have a concave upper surface 33.

While during tomographic imaging, the X-ray source 3 and the X-ray detector 7 may be displaced as shown in FIG. 2 in order to acquire a plurality of X-ray images under various tomographic angles α, there may also be other application modes.

For example, as indicated in FIG. 7, off-center screening images may be acquired while the X-ray detector 7 being positioned at one edge of the housing 5 and parallel to the upper supporting surface 13 of the housing 5. For example in MLO projection (Medio Lateral Oblique projection) in which the source-detector-arrangement is tilted, it may be important that an active area of the detector 7 is located close to the edge of the housing 7. For example, such position may be attained by moving the housing 5 accordingly.

An alternative application mode is shown in FIG. 8. In order to acquire screening images of e.g. a small breast 17 positioned on the supporting surface 13 of the housing 5, it may be advantageous to displace the detector 7 from a position adjacent to the upper surface 13 to a position (indicated by 7′) at an opposing lower surface 35 of the housing 5. With such parallel displacement of the detector 7, a spatial resolution and a DQE may be improved specifically for the case of small breasts to be examined. While in such specific application, the source-detector distance SID is increased by a distance ΔSID corresponding approximately to the depth of the housing 5, an orientation of the detector 7 remains substantially unchanged. Accordingly, for changing the source-detector distance SID, the moving mechanism 11 may radially translate the detector 7 without rotating it.

As indicated in FIG. 9, the detector 7 maybe provided with an anti-scatter-grid 37. The anti-scatter-grid 37 may be arranged in front of the detection surface 25 of the detector 7 and may be attached to the detector 7 such that it is moved/pivoted together with the X-ray detector 7. The anti-scatter-grid 37 may comprise lamellae 41 which are arranged approximately parallel to the X-ray beam 21 to be transmitted through the anti-scatter-grid 37 towards the detection surface 25. As the X-ray beam 21 may have a fan-like shape, lamellae 41 at an outer region of the anti-scatter-grid 37 may be arranged under a tilted angle while lamellae 41 at the center of the anti-scatter-grid 37 may be arranged perpendicular to the detection surface 25. Typically, the anti-scatter-grid 37 is designed for a specific source-detector distance SID. If used with another SID, the transmission of the anti-scatter-grid 37 may be reduced depending on the grid ratio. In mammography applications, this ratio is typically about 4. Accordingly, changing the SID depending on the tomographic angle α may not be ideal, but for small changes as provided in the proposed tomographic system, such influence should be negligible. Furthermore, specific detector calibration may improve remaining homogeneity issues.

In order to avoid stripes in the acquired X-ray images, the anti-scatter-grid 37 may be moved parallel to the detection surface 25 by a grid moving mechanism 39 (only schematically indicated) as indicated in FIG. 9 by the arrow. This may be typically a linear movement with a range of the order of 2 cm. When the anti-scatter-grid 37 is in an extreme position, it is stopped and moved in a reverse direction. The X-ray radiation from the X-ray source 3 may be interrupted during such stop of the anti-scatter-grid 37.

In conventional DBT systems, no anti-scatter-grid may be used as a grid-lamellae direction is usually incompatible with the angulation of the X-ray beam for different tomographic angles α. In the tomographic system proposed herein, an anti-scatter-grid 37 may be used advantageously in order to reduce noise induced by X-ray scattering and to thereby improve a signal-to-noise ratio in the acquired X-ray images. A turning point of the motion of the anti-scatter-grid may be set into the interval between two of the X-ray exposures. However, the exposure time of each individual X-ray projection image may be low (up to 25 times shorter than for a single screening image), so the motion blur may be limited and might not be enough. Some stripes induced by the anti-scatter-grid 37 may remain. However, even grid visibility with a non-moving anti-scatter-grid may be accepted in a raw image as it may be removed using for example image processing methods in the FFT (Fast Fourier Transformation) domain. As an alternative option, a grid filter may be used within the position space.

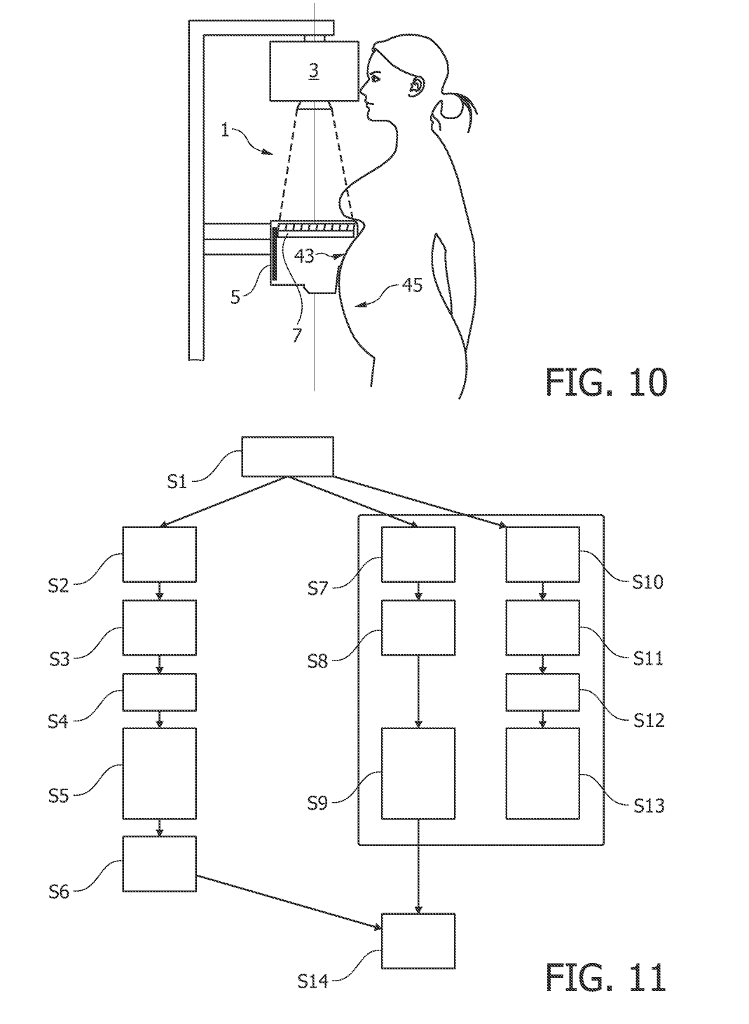

In order to be compatible with the conventional mammography screening systems, the proposed tomographic mammography system may be specifically adapted as shown in FIG. 10. For example, when acquiring a screening X-ray image of a breast 17 of a heavy woman, there may be a problem that the belly 45 of the heavy woman may interfere with the large housing 5 of the X-ray detector 7 of the proposed tomographic system 1. For such specific application, the housing 5 may be provided with a flexible front cover 43 which allows to resiliently deform upon contact with the patients belly 45. Accordingly, for screening applications in which the detector 7 is positioned directly underneath and parallel to the upper supporting surface 13 of the housing 5, an inside deformation of the flexible cover 43 does not interfere with the X-ray detector 7 as the lower portion of the housing 5 is basically empty in such screening applications. However, it is to be noted that for tomosynthesis applications in which the detector 7 is pivoted within the entire volume of the housing 5, a large volume of the detector housing 5 may not be avoided such that discomfort may apply for a heavy patient due to interference of the belly 45 with the large volume housing 5.

An operation mode of the proposed tomosynthesis system will be explained with reference to the flow-chart shown in FIG. 11. After starting DBT acquisition (step S1), a motion control unit is initiated (S2) and controls an angular movement α of the X-ray source and the X-ray detector (S3). Simultaneously or subsequently, an adequate change of the source-detector distance ΔSID is calculated (S4) and a radial translational movement of the detector is controlled (S5). Then all data on rotation cc and translation ΔSID are stored together with the respective images, e.g. in a header of an image (S6).

For each image acquisition at a respective tomographic angle α, the block on the right-hand side of FIG. 11 is repeated. The X-ray source is controlled (S7) and generates an X-ray flash (S8). The X-ray detector is triggered and read out (S9). Simultaneously, a movement of the anti-scatter-grid is controlled (S10) and the grid is moved linearly (S11). At an extreme position, the grid is stopped (S12) while an X-ray emission from the X-ray source is interrupted and the grid direction is inversed for a next X-ray flash (S13).

Finally, the acquired X-ray image data are saved and a resulting three-dimensional image of the female breast may be generated from the plurality of two-dimensional projection images acquired under various tomographic angles α.

Briefly summarizing, a novel tomosynthesis mammography system has been described which allows improved tomography with higher spatial resolution and an increased field of view. Tomographic angles larger than 2×45° may be feasible. The X-ray beam always impinges perpendicular to the detector such that an anti-scatter-grid can be used in order to improve contrast resolution. A driving force behind the innovation was to find a geometry which allows these improvements but keeps compatible with regular screening mode. Also stereotactic (guided) biopsy may be possible. A basic idea is to pivot the detector within a large housing having a flat or slightly curved upper surface simultaneously serving as a supporting surface for the female breast to be examined. In the pivoting movement, the detector is displaced translational along an axis normal to the detection surface of the detector while being rotated in accordance with a tomographic angle. With the proposed system, an x-y-resolution may be almost as good as in conventional screening mammography systems while a z-resolution may be somewhere between conventional DBT systems with fixed detector and breast computer tomography systems.

It should be noted that the term “comprising” does not exclude other elements or steps and that the indefinite article “a” or “an” does not exclude the plural. Also elements described in association with different embodiments may be combined. It should also be noted that reference signs in the claims shall not be construed as limiting the scope of the claims.

### List of Reference Signs

1 Tomography system
3 X-ray source
5 Housing
7 X-ray detector
9 Frame
11 Moving mechanism
13 Upper surface
15 Support arrangement
17 Female breast
19 arcuate path of X-ray source
21 X-ray beam
23 Middle axis of X-ray beam
25 Detection surface
27 Edge of X-ray detector
29 Opposing edge of X-ray detector
31 Flat surface of housing
33 Concave surface of housing
35 Lower surface of housing
37 Anti-scatter-grid
39 grid moving mechanism
41 Lamellae
43 Flexible front cover
45 Belly

1. A tomosynthesis system (1) for acquiring -a 3-dimensional image of an object (17), the system comprising:
an X-ray source (3);
an X-ray detector (7);
a support arrangement (15);
a moving mechanism (11);
wherein the X-ray source (3) and the X-ray detector (7) are adapted for acquiring a plurality of X-ray images while irradiating the object with an X-ray beam (21) from a plurality of tomographic angles α;
wherein the support arrangement (15) is adapted to support the object (17) during operation of the tomosynthesis system;
wherein the moving mechanism (11) is adapted to pivot the X-ray detector (7) in positions such that for each tomographic angle α a detection surface (25) of the X-ray detector (7) is oriented to be substantially perpendicular to the X-ray beam (21); and
wherein the moving mechanism (11) is adapted to move the X-ray detector (7) in positions such that a distance (SID) between the X-ray source (3) and the detector (7) increases with increasing tomographic angle α.

2. The tomosynthesis system of claim 1, wherein the moving mechanism (11) is adapted to move the X-ray detector (7) such that an increase of the distance (SID) between the X-ray source (3) and the detector (7) is proportional to the tangent of the tomographic angle α.

3. The tomosynthesis system of claim 1, further comprising a housing (5) enclosing the X-ray detector (7), wherein dimensions of the housing (5) are sized and the moving mechanism (11) is adapted such that for all positions to which the X-ray detector (7) may be moved by the moving mechanism (11) the housing (5) encloses the X-ray detector (7).

4. The tomosynthesis system of claim 3, wherein the housing (5) comprises a flat or concave surface (31; 33) forming the support arrangement (15) for supporting the object (17) during operation of the tomosynthesis system.

5. The tomosynthesis system of claim 4, wherein the flat or concave surface (31; 33) of the housing (5) forms the only X-ray absorption surface within an optical path between the X-ray source (3) and the X-ray detector (7).

6. The tomosynthesis system of claim 4, wherein the moving mechanism (11) is adapted to pivot and move the X-ray detector (7) such that for all tomographic angles α one edge (27) of the X-ray detector (7) is positioned adjacent to the flat or concave surface (31; 33) of the housing (5).

7. The tomosynthesis system of claim 3, wherein the housing (5) comprises a flexible front cover (43).

8. The tomosynthesis system of claim 1, further comprising an anti-scatter grid (37) arranged between the X-ray detector (7) and the support arrangement (15).

9. The tomosynthesis system of claim 8, wherein the anti-scatter grid (37) is attached to the x-ray detector (7).

10. The tomosynthesis system of claim 8, further comprising a grid moving mechanism (39) for moving the anti-scatter grid (37) parallel to the detection surface (25) of the X-ray detector (7).

11. The tomosynthesis system of claim 1, wherein the moving mechanism (11) is further adapted to move the detector (7) in order to increase the distance (SID) between the X-ray source (3) and the detector (7) while an orientation of the detector (7) remains fixed.

12. The tomosynthesis system of claim 1, wherein the X-ray source (3) and the X-ray detector (7) are adapted to acquire X-ray images within a range of tomographic angles of more than +/−25°.
